(19) United States
(12) Patent Application Publication
(10) Pub. No.: US 2023/0204510 A1
(43) Pub. Date: Jun. 29, 2023

(54) OPTICAL DETECTOR

(71) Applicant: ams-OSRAM AG, Premstaetten (AT)

(72) Inventors: Boon Chong Cheah, Eindhoven (NL); Alexander Volk, Kainbach bei Graz (AT); David Gamperl, Hartberg (AT); Massimo Mascotto, Milano (IT); Erik Jan Lous, Veldhoven (NL)

(21) Appl. No.: 17/925,714

(22) PCT Filed: May 18, 2021
(86) PCT No.: PCT/EP2021/063118

§ 371 Date: Nov. 16, 2022

(30) Foreign Application Priority Data
May 22, 2020 (GB) 2007683.2

(51) Int. Cl. G01N 21/64; G01J 3/42

(52) U.S. Cl. CPC G01N 21/6408; G01J 3/42; G01J 2001/446

(57) ABSTRACT

An optical detector (1) on an application specific integrated circuit (ASIC) comprises at least one photodiode (5) for receiving incident light and configured to provide at least one diode signal, a modulator (2) configured to provide an AC drive signal and to provide a reference signal associated with the AC drive signal; and a lock-in amplifier (6) configured to receive said at least one diode signal from said at least one photodiode (5) and to receive the reference signal from the modulator (2), and to determine at least one of a phase and an amplitude of said at least one diode signal using the reference signal.

### FIELD

The present disclosure relates to optical detectors.

### BACKGROUND

Fluorescence spectroscopy is one of the most sensitive detection techniques to quantify molecules. This is because the measurement is done in a dark background and the light source is incident with an off-axis angle. Furthermore, fluorescence intensity is not dependent on the path length of the sample, which is a limitation to absorbance spectroscopy. There are two methods of doing fluorescence spectroscopy, time resolved and phase modulation.

In a conventional phase modulation system, the sample is illuminated with a modulated light source where the frequency is chosen based on the lifetime of the fluorophores in the sample. The light from the sample is detected by a photomultiplier tube (PMT) and the phase and amplitude of the output signal are compared to those of the light modulation signal.

Fluorescence lifetime is one of the most robust fluorescence parameters and is used, for example, in applications where it is necessary to discriminate against the high background fluorescence from biological samples. The fluorescence lifetime is the average decay time of a fluorescent molecule from its excited state to ground state by emitting photons. As can be seen in FIG. 1, the population of fluorophores are excited by an intensity=I₀ at t=0. The fluorophores will decay from their excited state to the ground state over a period. Following the equation:

$$I ( t ) = {I}_{0} {e}^{\frac{- t}{τ}} \qquad ( 1 )$$

Where I₀ is the original value of the excitation state and τ is the lifetime. The lifetime is defined as the time of the excited intensity to decay to 1/e or 36.79% of its original value.

### SUMMARY

The inventors have appreciated that at least some of the problems associated with known methods of spectroscopy can be overcome by using lock-in detection on an application specific integrated circuit (ASIC) chip. Lock-in detection is a method that is capable of extracting signal amplitudes and phases in extremely noisy environments. The working principle of a lock-in measurement is by extracting a signal at a defined frequency that is identical to the reference modulated frequency and eliminating all other frequency components. This approach utilises homodyne detection and band-pass filtering to measure the signal's amplitude and phase that is relative to the reference frequency. By doing so, the signal of interest can be measured accurately and a high SNR can be achieved. FIG. 2 illustrates how a lock-in amplifier is used to extract the amplitude and phase of a noisy signal Vₛ(t) by using a reference signal Vᵣ(t).

Lock-in detection could increase SNR in spectroscopic measurements and be used to perform fluorescence lifetime measurements. A disadvantage of existing lock-in detection is the bulky electronics. Existing systems have bulky bench top equipment with discrete components that are expensive.

According to a first aspect of the present invention there is provided an optical detector on an application specific integrated circuit (ASIC) comprising at least one photodiode for receiving incident light and configured to provide respective diode signals; a modulator configured to provide an AC drive signal and to provide a reference signal associated with the AC drive signal; and a lock-in amplifier configured to receive the diode signals from the at least one photodiode and to receive the reference signal from the modulator, and to determine a phase and/or an amplitude of the diode signals using the reference signal. The modulator is typically a light source modulator configured to drive a light source with the AC drive signal. For some applications the modulator can be configured to drive a heating element (e.g. a heating coil) coupled to the sample or to apply a voltage directly across the sample in order to excite the sample and cause it to luminesce.

The optical detector can be included as a single integrated system to obtain fluorescence lifetime using lock-in detection and phase modulation fluorescence with improved SNR. For spectroscopic measurements, the SNR may be improved by a few orders of magnitude compared to a DC optical detector (i.e. an optical detector without frequency modulation or lock-in detection). Compared to existing lock-in detection systems, the optical detector has the advantage of fewer standalone or individual components (such as PMTs), which allows the optical detector to be made more compact, improve alignment robustness, and can especially reduce noise. For example, the product package of the ASIC chip comprising the optical detector may have dimensions in the following ranges: Width=2 mm to 5 mm; Length=2 mm to 5 mm; and Height=0.2 mm to 2 mm. The product package may comprise a light source such as an LED, or the light source can be provided separately.

The optical detector is typically a spectrometer. One or more of the at least one photodiodes typically comprise a colour filter, to be sensitive to a particular colour (i.e. frequency range). For example, dichroic filters having a FWHM of about 5 nm to 40 nm may be used. The at least one photodiode may comprise a mixture of filtered photodiodes and clear (un-filter) photodiodes. Two or more photodiodes may comprise the same colour filter. The ASIC chip may integrate filters into standard CMOS silicon via Nano-optic deposited interference filter technology. Using the optical detector with specific dichroic filters can allow the system to discriminate the measurement of the specific wavelength of the fluorescence emission, while rejecting any stray light coming from the excitation light source.

The optical detector typically comprises an array of photodiodes. That is, the at least one photodiode is typically a plurality of photodiodes arranged in an array. The amplifier of the optical detector may comprise a multiplexer configured to multiplex the diode signals from the plurality photodiodes. For example, the plurality of photodiodes may be an 8×8 array, providing 64 individual signals, while the ASIC may only comprise, for example, 16 physical channels to process the signals. The multiplexer can then multiplex the 64 signals to 16 signals, which can then be processed in parallel on the 16 channels. Each photodiode can be individually lock-in detected to determine its signal strength (amplitude) and phase. Alternatively, signals from groups of similar photodiodes (e.g. having the same colour filter) may be processed as one signal, wherein it is assumed that the phase of signals from photodiodes within the group is substantially the same.

The optical detector may use analogue mixing (i.e. mixing of analogue signals) of the pixel diode signal with the driver reference, thereby determining via normal lock-in detection the amplitude and phase of the (each) pixel diode signal. The lock-in amplifier may comprise: a mixer configured to mix the reference signal with an output from the multiplexer to provide demodulated signals; a second multiplexer coupled to the first multiplexer and configured to multiplex the demodulated signals; and one or more analogue to digital converters (ADCs) configured to convert the demodulated signals to digital signals. The amplifier provides analogue mixing and lock-in detection, by demodulating the analogue signals from the diodes before they are digitizing by the ADCs.

Alternatively, the optical detector may be configured to use digital mixing (i.e. mixing of digital signals) of the photodiode signal with the driver reference to determine, via digital lock-in detection, the amplitude and phase of the (each) pixel diode signal. In this case, the amplifier may comprise one or more analogue to digital converters (ADCs) configured to convert an output from the multiplexer into digital signals; a mixer configured to mix the digital signals with the reference signal to provide demodulated signals; and a second multiplexer coupled to the first multiplexer and configured to multiplex the demodulated signal. The lock-in amplifier thereby provides digital demodulation and digital lock-in detection.

The first and a second multiplexers can be coupled to select each photodiode signal (or each group/set of photodiode signals) for demodulation by the mixer and then to bring the demodulated signal to the data buffer or MCU. The optical detector can comprise one or more further lock-in amplifiers connected in parallel and configured to determine the phase and/or amplitude of signals using the reference signal. The set of MUX, MIX, MUX, and ADC can be made on the ASIC in multitudes (duplo, triple, . . . multiple) in parallel to increase the speed of measurement and data analysis.

The light source may comprise at least one of a light emitting diode (LED), a lamp (e.g. a light bulb), and/or a vertical cavity surface emitting laser (VCSEL). The light source modulator may comprise a programmable maximum duty cycle and frequency oscillator, or may comprise an analogue current/amplitude modulator. The light source modulator may be configured to perform pulse width modulation (PWM). The light source modulator can provide an AC drive signal to the light source in order to provide modulated light. The AC may optionally have a DC-offset. The AC drive signal can be one of a sine wave, a square wave, and a triangular wave. In principle having AC-output from zero-max and may have an offset. Other waves, including stochastic, pseudo-random and quasi random drive signals may also be used, since the lock-in amplifier is provided with an associated reference signal, which enables demodulation. The AC drive signal may have a frequency in the range of typically 2 Hz to 10 MHz, and the reference signal has the same frequency as the drive signal. The large frequency range provided by the light source modulator may be beneficial for spectroscopic analysis of a large variety of samples (e.g. different fluorophores having different fluorescence lifetimes). In absorbance and reflectance mode, the optical detector can provide broad spectral ranges for identifying compounds.

The components of the optical detector, i.e, the light source modulator, the photodiodes and lock-in amplifier, are integrated on a single ASIC chip, such as an integrated CMOS chip. The ASIC can be configured to be powered by a supply voltage (VDD) in the range of 1.6 V to 2.0 V, for example 1.8 V. The low voltage can reduce power consumption. The small form factor of the ASIC may therefore be especially suited for Point-of-Care settings, wearables and battery driven, low power devices. It can have improved cost, low noise through minimized parasitics, and a small form factor.

In addition to increasing SNR by eliminating noise, the optical detector can allow the differentiation of different fluorescence lifetimes. This may be particularly useful in medical devices, as general human biofluid samples have different auto-fluorescence emission when excited in the UV to visible range. Using a phase modulation technique with the optical detector, the different phase shifts and modulation shifts provide the fluorescence lifetime. Therefore, the auto-fluorescence can be determined and only the corresponding phase/modulation shift to the target fluorophore picked out in the measurements. Furthermore, the optical detector can be used in a multiplexing technique, when more fluorophores with the same emission wavelength are used. The phase modulation technique allows the differentiation of the different fluorophores.

The invention integrates lock-in detection with photodiodes into a single ASIC chip being a spectral sensor chip. Compared to dc-spectral sensors, the invention can add sensitivity and higher dynamic range. The chip can provide the modulation in the driver currents for illumination devices such as LEDs, (miniaturized) lamps and VCSELs, whereas classically choppers where applied. Note that each pixel of the diode detector array is demodulated for response in amplitude and phase. Each pixel diode may contain an optical filter or not.

According to a second aspect of the invention there is provided a system for performing spectroscopic measurements of a sample comprising: means for exciting the sample; and an optical detector according to the first aspect of the invention arranged such that the at least one photodiode receives light from the sample when in use. The means for exciting the sample may comprise one of a light source, a heating element (e.g. current carrying coil) and electrodes for applying a voltage across the sample.

The system may further comprise a sample holder for holding the sample. The sample holder may comprise a lateral flow test strip comprising a test line, wherein the light source is configured to illuminate the test line. The optical detector can then be arranged such that the at least one photodiode receives light reflected from the test line or emitted by the test line.

The system can be configured to be used in applications measuring at least one of reflectance, transmission/absorbance, and fluorescence or luminescence.

The optical detector can be housed in a product package having dimensions of about 2 mm×3 mm×1 mm (width×length×height), whereby ‘about’ indicates a variance of 10%. The miniaturised nature of the chip allows for a very small product package compared to existing solutions. The means for exciting the sample (e.g. light source, heater, voltage source) may be located outside the product package and is driven by the ASIC. For example, the light source may be provided as a separate module that is connected to the ASIC inside the product package.

According to another aspect of the invention there is provided a method of performing spectroscopic measurements using an optical detector according to the first aspect. The step of using the optical detector may comprise driving a light source with the AC drive signal from the light source modulator; illuminating a sample with the light source; receiving with the at least one photodiode light reflected by or emitted from or transmitted through the sample; and using the lock-in amplifier to determine the phase and/or amplitude of the light received by the at least one photodiode. The step of using the lock-in amplifier may comprise mixing the at least one diode signal from the at least one diode with the reference signal from the light source modulator. One or more of the at least one photodiode may have an optical filter for a specific wavelength and with a specific bandwidth.

According to a further aspect of the invention there is provided a method of determining the amplitude and/or phase of light using an optical detector on an application specific integrated circuit (ASIC), comprising driving a light source with an AC drive signal from a light source modulator; illuminating a sample with the light source; receiving with at least one photodiode light reflected or emitted from the sample; receiving at a lock in amplifier at least one diode signal from the at least one diode and a reference signal associated with the AC drive signal from the light source modulator; and using lock-in detection to determine the phase and/or amplitude of the at least one diode signal from the at least one diode signal and the reference signal.

### BRIEF DESCRIPTION OF THE DRAWINGS

FIG. 1 is a graph illustrating the decay of fluorescence intensity over time after an initial excitation;

FIG. 2 shows a schematic diagram of a lock-in amplifier;

FIG. 3 is a graph illustrating the phase shift and change in amplitude of an emitted signal relative to an excitation signal;

FIG. 4 is a schematic diagram of a chip with a spectrometer according to an embodiment configured for analogue lock-in detection;

FIG. 5 is a schematic diagram of a chip with a spectrometer according to an embodiment configured for digital lock-in detection;

FIG. 6 is a schematic diagram of a system according to an embodiment for performing a lateral flow test using an optical detector;

FIG. 7 is a schematic diagram of a system according to an embodiment wherein the system is configured to operate in an absorbance mode;

FIG. 8 is a schematic diagram of a system according to another embodiment wherein the system is configured to operate in an absorbance mode;

FIG. 9 is a schematic diagram of a system according to an embodiment operating in fluorescence mode;

FIG. 10 is a schematic diagram of a system according to an embodiment operating in luminescence mode;

FIG. 11 is a schematic diagram of a system according to an embodiment operating in reflectance mode; and

FIG. 12 is a schematic diagram of a system according to an embodiment operating in absorbance mode.

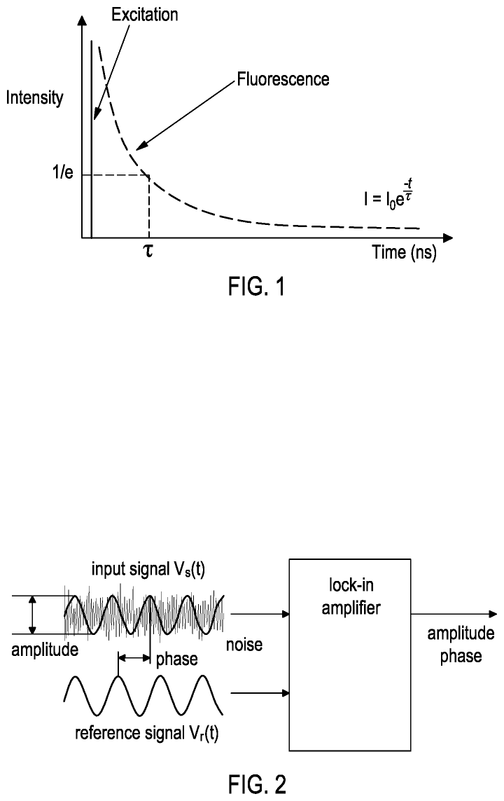

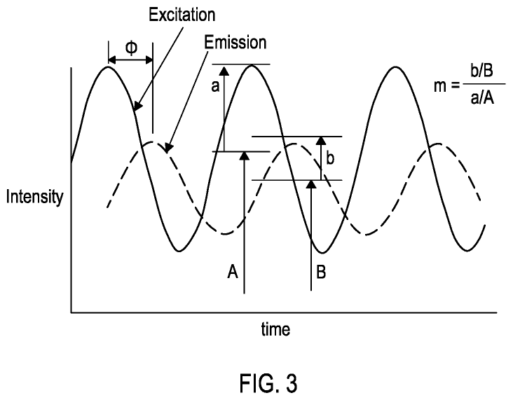

### DETAILED DESCRIPTION

There are two methods of performing time-resolved fluorescence measurements, time domain and frequency domain. In the time domain, the sample with fluorophores is excited with a short pulse of light and the bandwidth of the pulse is shorter than T. Then, the time dependent intensity is measured over a period until 1/e of the original value at t=0 to obtain the lifetime or taking the slope of a plot log I(t) vs t.

The other method of measurement is frequency domain or phase modulation technique. In this technique, the sample with fluorophores is excited with an intensity modulated light source and normally in the form of a sine wave to avoid harmonic frequencies that could generate noise. The intensity of the light source has to be modulated at a frequency that is comparable to the reciprocal of the lifetime r. By doing this, the emission of the fluorescence is forced to respond at the same modulation frequency. However, due to the lifetime of the fluorescence, there is a delay in time relative the modulated excitation. This delay can be seen in FIG. 2 as a phase lag, Φ and can be used to calculate the fluorescence lifetime:

$${τ}_{ϕ} = \frac{1}{ω} tan ϕ \qquad ( 2 )$$

In addition, another effect due to fluorescence lifetime is the peak-to-peak height of the emission relative to modulated excitation,

$$m = \frac{b / B}{a / A} .$$

The decrease in modulation is because some of the fluorophores that are excited are still emitting photons when the excitation is at a minimum, which is due to the quantum yield of the common fluorophores is less than 100%. This effect is named demodulation and can also be used to calculate the fluorescence lifetime:

$${τ}_{m} = \frac{1}{ω} {( \frac{1}{{m}^{2}} - 1 )}^{\frac{1}{2}} \qquad ( 3 )$$

In addition, in medical devices that normally measure biological samples that have auto-fluorescence in the visible range, using a phase modulation technique, each separate lifetime components can be separated to obtain the correct signal from the designated fluorophores that are used for detection.

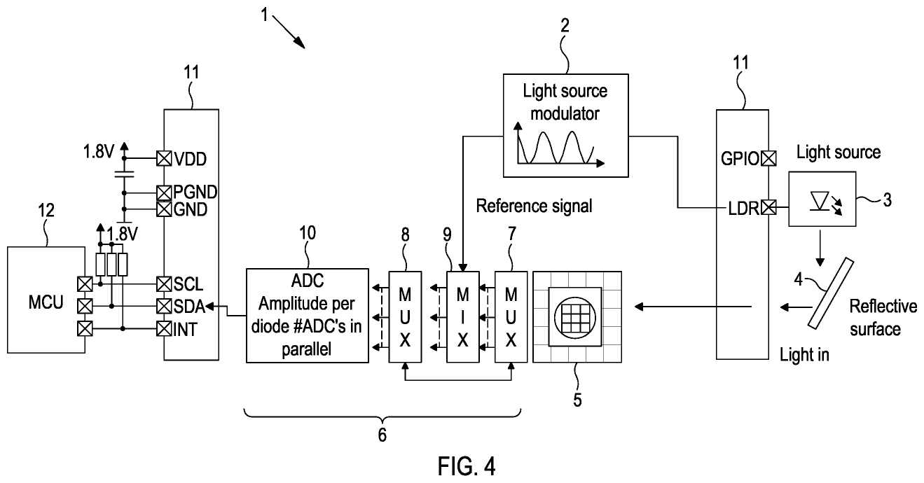

FIG. 4 illustrates a first embodiment of an optical detector being a spectrometer 1 configured to perform analogue mixing and lock-in detection. The spectrometer 1 is located on an ASIC chip formed in CMOS process. The spectrometer 1 comprises a light source modulator 2, which provides a sinusoidal drive signal to an LED 3. The light source modulator 2 may also be configured to provide other types of signals of arbitrary shape, such as a block pulse or triangle. The LED 3 illuminates a sample 4, which reflects light onto an array of photodiodes 5. At least some of the photodiodes 5 comprise colour filters for selective sensitivity in a frequency range. The colour filters are integrated on the same chip as the photodiodes 5, for example located in the back end stack of the CMOS chip. The photodiodes 5 output respective diode signals, which are received by the lock-in amplifier 6. A plurality of lock-in amplifiers 6 can be used in parallel to increase processing speed. The lock-in amplifier 6 uses a reference signal, having the same frequency as the drive signal, from the light source modulator 2 to demodulate the signals from the photodiodes 5 in order to determine the phase and amplitude of the signals. The lock-in amplifier 6 comprises two multiplexers 7 and 8, a mixer 9 and a plurality of ADCs 10 in parallel. In this embodiment the first multiplexer 7 is used to individually port each diode signal from the array 5 to the mixer 9, where the analogue signal is demodulated in its amplitude and phase. Hence, the spectrometer 1 of the first embodiment is configured to perform analogue mixing and lock-in detection by processing the analogue signals. The chip comprises various input and/or output pins 11 for using the spectrometer, and a micro control unit 12 (MCU) for controlling the spectrometer 1 and processing data provided by the spectrometer 1. The second multiplexer 8 may bring the demodulated signals to the MCU 12 or to a data buffer. In an alternative embodiment, the MCU 12 is external and not integrated on the ASIC.

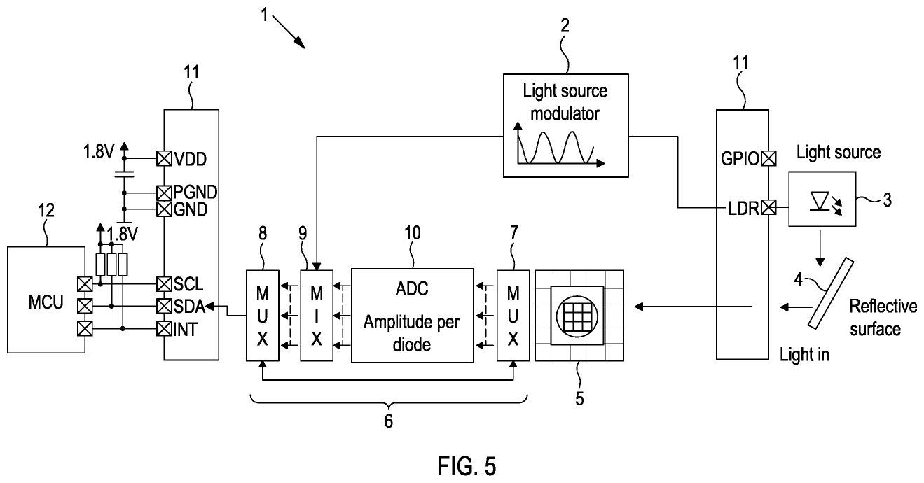

FIG. 5 illustrates an optical detector being a spectrometer 1 on an ASIC chip formed in a CMOS process according to a second embodiment. Features in FIG. 5 similar to those of FIG. 4 have been given the same reference numerals for clarity and are not intended to be limiting. The spectrometer 1 comprises a light source modulator 2 for providing a sinusoidal drive signal to a light source 3, being an LED. The light source modulator 2 can also be configured to provide a drive signal to the light source 3 having a different, non-sinusoidal, shape. The LED 3 is arranged to illuminate a sample 4, which reflects light onto an array of photodiodes 5 (the spectrometer may also be used in transmission/absorbance mode, and in fluorescence mode by appropriate arrangement of the light source). At least some of the photodiodes 5 comprise colour filters for selective sensitivity in a frequency range. The colour filters are integrated on the same chip as the photodiodes, for example located in the back end stack of the CMOS chip. The photodiodes 5 output respective diode signals, which are received by the lock-in amplifier 6. A plurality of lock-in amplifiers 6 can be used in parallel to increase processing speed. The lock-in amplifier 6 uses a reference signal from the light source modulator 2 to demodulate the signals from the photodiodes in order to determine the phase and amplitude of the signals. The lock-in amplifier 6 comprises two coupled multiplexers 7 and 8, a mixer 9 and an ADC 10. A plurality of multiplexers 7 and 8 may be used in parallel together with a respective plurality of ADCs 10. The first multiplexer 7 receives the diode signals from the photodiode array 5 and reduces the number of channels so that the signals can be digitized by the ADCs 10 (one ADC per channel). The digital signals are then demodulated (per channel) in phase and amplitude by the mixer 9 using the reference signal provided by the light source modulator 2. The second multiplexer 8 outputs the phase and amplitude of each diode signal from the photodiodes 5 to the correct destination (e.g. in a data buffer or to the MCU). The spectrometer 1 of the second embodiment is configured to perform digital mixing and digital lock-in detection by processing the digitized signals. The chip comprises various input and/or output pins 11 for using the spectrometer, and a micro control unit 12 (MCU) for controlling the spectrometer 1 and processing data provided by the spectrometer 1.

In one embodiment, an optical detector has 11 channels for spectral identification and colour matching applications used in mobile devices. The optical detector comprises a light source modulator for driving a light source and a lock-in amplifier connected to the photodiodes and connected to the light source modulator for demodulating the diode signals. The optical detector may be configured to measure the spectral response defined in the wavelengths from approximately 350 nm to 1000 nm. Six channels can be processed in parallel by independent ADCs while the other channels are accessible via a multiplexer. Eight optical channels associated with 16 photodiodes (4×4 photodiode array) cover the visible spectrum (VIS). One channel can be used to measure near infra-red (NIR) light, and another channel is associated with a photodiode without filter (“clear”). The optical detector may also integrate a dedicated channel to detect 50 Hz or 60 Hz ambient light flicker. The flicker detection engine can also buffer data for calculating other flicker frequencies externally. The NIR channel in combination with the other VIS channel may provide information of surrounding ambient light conditions (light source detection). The optical detector can be synchronized to external signals via a general purpose input/output (GPIO) pin.

In one embodiment, the ASIC chip integrates filters into standard CMOS silicon via Nano-optic deposited interference filter technology. A built in aperture is provided to control the light entering the photodiode array. Control and spectral data access is implemented through a serial I²C interface. The device can have an ultra-low profile package with dimensions of 3.1 mm×2 mm×1 mm.

Embodiments of the optical detector can be implemented in a lateral flow test. A typical lateral flow test will have two measureable lines, test and control lines. The test line gives information on the different concentrations of analytes as a function of fluorescence intensity. Normally, to measure this in a lateral flow test, a reflection mode is used.

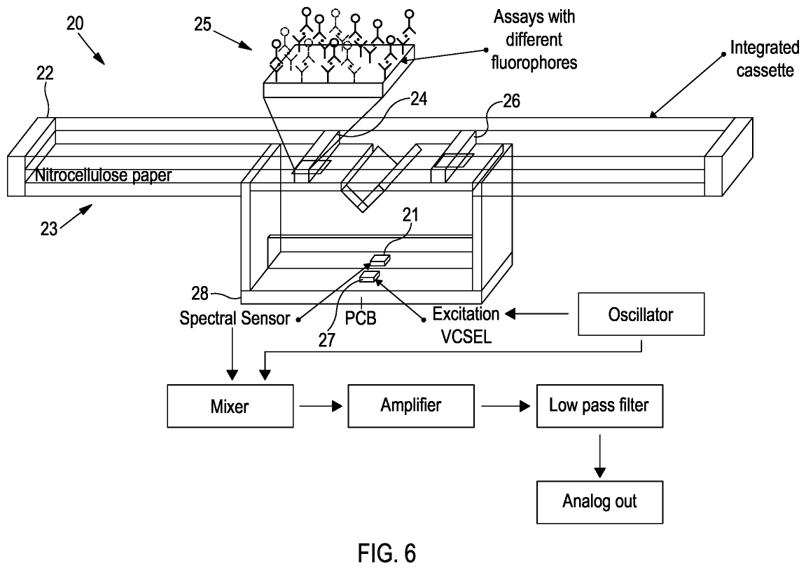

FIG. 6 shows a schematic diagram of a system 20 for performing a lateral flow test according to an embodiment. The system 20 is a phase modulation fluorescence measurement system, using a spectrometer 21 according to an embodiment. The system 20 comprises a lateral flow test strip 22 comprising nitrocellulose paper 23, a test line 24 comprising assays with different fluorophores 25, and a control line 26. The system 20 further comprises the spectrometer 21, which is fixed at least in one dimension relative to the lateral flow test strip 22 to operate in a reflectance and fluorescence mode. The photodiodes are arranged to receive light reflected from the test line 24. The chip, comprising the spectrometer 21, is connected to the light source 27 via a PCB 28. The light source modulator comprises an on-board oscillator used to modulate the output of an excitation VCSEL to match the reciprocal of a known lifetime of the target sample fluorophore. Each photodiode is connected to an on-chip mixer that is connected with the reference frequency of the oscillator, for demodulation of the signal. Subsequent amplification and filtering, reveals the amplitude (and phase) as a signal. This method can increase the SNR of a lateral flow test in an off-axis measurement scheme. Using equations 1 to 3, the amplitude and phase of the output can be obtained.

In general, embodiments of the optical detector can be advantageously used for bio-diagnostics in lateral flow tests. Embodiments can improve the sensitivity, especially when configured to operate in fluorescence mode. The small package size and improved robustness can enable implementation of the optical detector in hand held systems, which has previously not been possible. The detection can be done in the frequency domain as well as in the time domain.

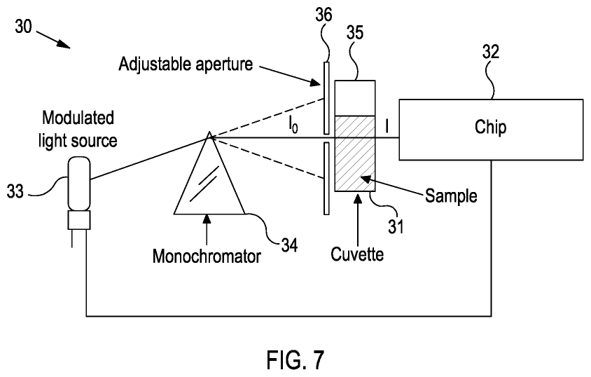

FIG. 7 shows a schematic diagram of a system 30 according to an embodiment for testing a sample 31 in absorbance mode, wherein an optical detector 32 and light source 33 are arranged such that the sample 31 can be located between the light source 33 and the photodiodes of the optical detector 32. The system 30 further comprises a monochromator 34 for filtering the light from the light source 33, a sample holder 35 being a cuvette 35 for holding the sample 31, and an adjustable aperture 36 for adjusting the intensity of light transmitted to the sample 31.

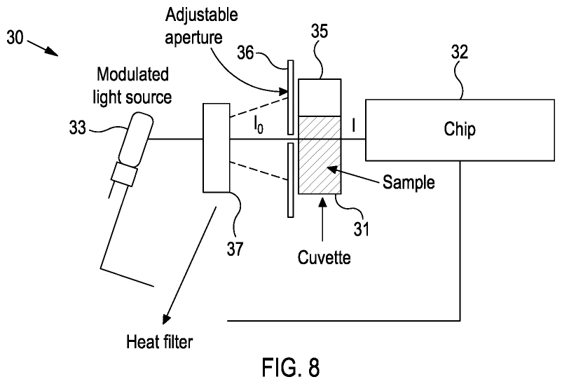

FIG. 8 shows a schematic diagram of a system 30 according to an embodiment for testing a sample 31 in absorbance mode similar to the system illustrated in FIG. 7. The photodiodes of the optical detector 32 comprise filters (not shown), such that the optical detector is a spectrometer 32. In this embodiment, because of the filters of the photodiodes, a monochromator is not required. The system 30 comprises a heat filter 37, for blocking unwanted frequencies in the IR and/or NIR spectrum.

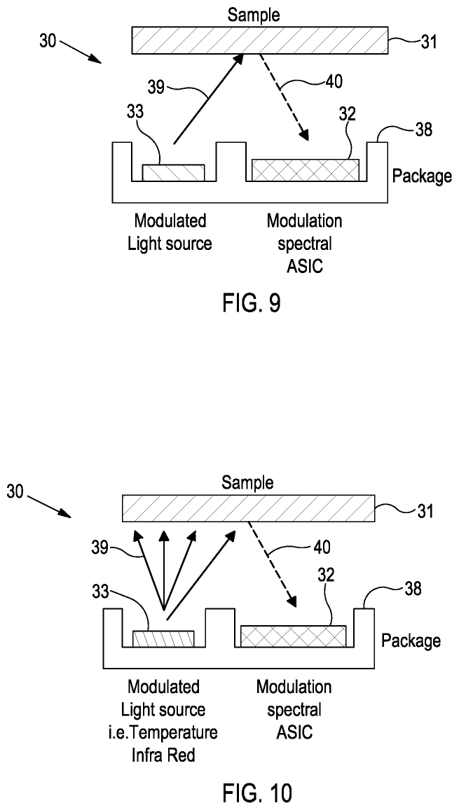

FIGS. 9 to 12 illustrate four different operation modes being fluorescence, luminescence, reflectance and absorbance respectively of an optical detector 32 according to one or more embodiments.

FIG. 9 is a schematic diagram of a system 30 for performing spectroscopic measurements with one or more diode pixels with integrated filter of a sample 31 in fluorescence mode. The system 30 comprises a product package 39 comprising a spectrometer 32 on an ASIC chip and the light source 33, wherein the spectrometer 32 is connected to the light source 33 in order to drive the light source with an AC drive signal. The light source 33 is arranged relative to the sample to illuminate the sample 31 with modulated light 39. The sample contains one or more fluorophores, which fluoresce and thereby emit light 40 that is received by the spectrometer 32. The modulated light 39 emitted by the light source 33 and the fluoresced light 40 emitted by the sample may in general have different wavelengths. Embodiments of the optical detector can be used for miniaturized fluorescence measurements, for example to perform bio-diagnostics using a lateral flow test.

FIG. 10 is a schematic diagram of a system 30 for performing spectroscopic measurements of a sample 31 in luminescence mode. The system 30 comprises a product package 39 comprising a spectrometer 32 on an ASIC chip and the light source 33. The light source 33 may emit light having a wavelength in the IR or NIR spectrum and is arranged relative to the sample to illuminate the sample 31 with modulated light 39, causing the sample to modulate in temperature. Other means for exciting the sample may also be used. For example, temperature modulation may be induced by means of an electrical current through a conducting coil around the sample 31. The sample 31 absorbs the light 39 (or heat) and in response emits light 40 through luminescence. The spectrometer 32 is arranged to receive the light 40 emitted by the sample 31. In another embodiment, the modulator is configured to apply a varying voltage directly across the sample 31 via electrodes, wherein the luminescence of the sample 31 is modulated by the applied voltage (so called electroluminescence).

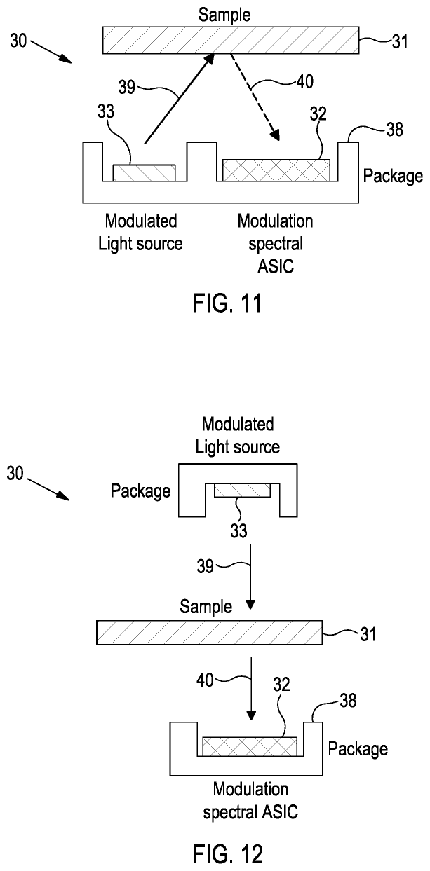

FIG. 11 is a schematic diagram of a system 30 for performing spectroscopic measurements of a sample 31 in reflectance mode. The system 30 comprises a product package 39 comprising a spectrometer 32 on an ASIC chip and the light source 33, wherein the spectrometer 32 is connected to the light source 33 in order to drive the light source 33 with an AC drive signal. The light source 33 is arranged relative to the sample to illuminate the sample 31 with modulated light 39 at an angle. The sample 31 reflects light from the light source 33 at an angle such that the reflected light 40 is incident upon the photodiodes of the spectrometer 32.

Embodiments of the optical detector can be used for miniaturized reflectance applications. For example, such a spectrometer can be used for color measurements, for example, to measure skin tone and/or to measure moisture of samples e.g. grain, beans, etc. The spectrometer can provide faster results and shorter integration time. The spectrometer may also be used for measuring smaller areas, which can be particularly useful for samples that are not homogeneous.

FIG. 12 is a schematic diagram of a system 30 for performing spectroscopic measurements of a sample 31 in absorbance mode. The system 30 comprises a product package 39 comprising a spectrometer 32 on an ASIC chip and the light source 33, wherein the spectrometer 32 and the light source 33 are arranged such that the sample 31 can be located between them. The spectrometer 32 is connected to the light source 33 in order to drive the light source 33 with an AC drive signal. The light source 33 is arranged relative to the sample to illuminate the sample 31 with modulated light 39. The sample 31 blocks (e.g. absorbs or reflects) a portion of the incident light 39 and transmits another portion of light 40. The spectrometer 32 is arranged to receive the transmitted light 40.

Embodiments of the optical detector can be used for miniaturized scatter measurements, and can be used in a particle sensor and/or smoke sensor. The optical detector can provide an increased dynamic range and greater sensitivity in order to detect smaller concentrations of particles as well as smaller particles. The optical detector can be integrated in a small sensor module (e.g. due to the small form factor of the ASIC chip), which can make it particularly suitable for household appliances.

Other embodiments of the optical detector can be used for miniaturized Raman spectroscopy, for example to measure hydration.

An embodiment of the optical detector can be integrated in a vital sensor, configured to optically measure blood pressure with reduced noise compared to existing methods.

Although the invention has been described in terms of preferred embodiments as set out above, these embodiments are illustrative only and the claims are not limited to those embodiments. Those skilled in the art will be able to make modifications and alternatives in view of the disclosure which fall within the scope of the claims. Each feature disclosed or illustrated in the present specification may be incorporated in the invention, whether alone or in any appropriate combination with any other feature disclosed or illustrated herein.

1. An optical detector on an application specific integrated circuit (ASIC) comprising:
at least one photodiode for receiving incident light and configured to provide at least one diode signal;
a modulator configured to provide an AC drive signal and to provide a reference signal associated with the AC drive signal; and
a lock-in amplifier configured to receive said at least one diode signal from said at least one photodiode and to receive the reference signal from the modulator, and to determine at least one of a phase and an amplitude of said at least one diode signal using the reference signal.

2. An optical detector according to claim 1, wherein the modulator is a light source modulator configured to drive a light source with the AC drive signal.

3. An optical detector according to claim 1, wherein, when the at least one photodiode forms a plurality of photodiodes and the at least one diode signal forms a plurality of diode signals, each diode signal being provided by a respective photodiode, the amplifier comprises a multiplexer configured to multiplex the plurality of diode signals from the plurality of photodiodes into one or more groups, wherein the lock-in amplifier is configured to, for the or each group, determine the at least one of the phase and the amplitude.

4. An optical detector according to claim 3, wherein the amplifier further comprises:
a mixer configured to mix the reference signal with an output from the multiplexer to provide demodulated signals;
a second multiplexer coupled to the first multiplexer and configured to multiplex the demodulated signals; and
one or more analogue to digital converters (ADCs) configured to convert the demodulated signals to digital signals.

5. An optical detector according to claim 3, wherein the amplifier further comprises:
one or more analogue to digital converters (ADCs) configured to convert an output from the multiplexer into digital signals;
a mixer configured to mix the digital signals with the reference signal to provide demodulated signals; and
a second multiplexer coupled to the first multiplexer and configured to multiplex the demodulated signals.

6. An optical detector according to claim 4, wherein the first and second multiplexers are configured to select each photodiode signal and/or group of photodiode signals.

7. An optical detector according to claim 1 and comprising one or more further lock-in amplifiers connected in parallel and configured to determine the phase and/or amplitude of signals using the reference signal.

8. An optical detector according to claim 1, wherein the light source comprises at least one of a light emitting diode (LED), a lamp, and a vertical cavity surface emitting laser (VCSEL).

9. An optical detector according claim 1, wherein the light source modulator comprises a programmable maximum duty cycle and frequency oscillator.

10. An optical detector according to claim 1, wherein the light source modulator is configured to perform pulse width modulation (PWM).

11. An optical detector according to claim 1, wherein the AC drive signal is one of a sine wave, a square wave, and a triangular wave.

12. An optical detector according to claim 1, wherein the AC drive signal has a DC offset.

13. An optical detector according to claim 1, wherein the AC drive signal has a frequency in the range of 2 Hz to 10 MHz, and the reference signal has the same frequency as the drive signal.

14. An optical detector according to claim 1, wherein the ASIC is configured to be powered by a supply voltage (VDD) in the range of 1.6 V to 2.0 V.

15. An optical detector according to claim 1, wherein one or more of the at least one photodiode comprises a colour filter.

16. A system for performing spectroscopic measurements of a sample comprising:
means for exciting the sample; and
an optical detector according to claim 1 arranged such that the at least one photodiode receives light from the sample when in use.

17. A system according to claim 16, wherein the means for exciting the sample comprises a light source.

18. A system according to claim 17, and further comprising a sample holder for holding the sample wherein:
the sample holder comprises a lateral flow test strip comprising a test line;
the light source is configured to illuminate the test line; and
the optical detector is arranged such that the at least one photodiode receives light reflected from the test line or emitted by the test line.

19. A system according to claim 16, wherein the optical detector and the means for exciting the sample are arranged to measure at least one of reflectance, absorbance, fluorescence, and luminescence.

20. A system according to claim 16, wherein the ASIC is housed in a product package having dimensions of about 2 mm×3 mm×1 mm.

21. A system according to claim 20, wherein the means for exciting the sample is located outside the product package and is driven by the ASIC.

22. A method of performing spectroscopic measurements using an optical detector according to claim 1.

23. A method according to claim 22, wherein the step of using the optical detector comprises:
driving a light source with the AC drive signal from the light source modulator;
illuminating a sample with the light source;
receiving with the at least one photodiode light from the sample; and
using the lock-in amplifier to determine the phase and/or amplitude of the light received by the at least one photodiode.

24. A method according to claim 23, wherein the step of using the lock-in amplifier comprises mixing the at least one diode signal from the at least one diode with the reference signal from the light source modulator.

25. A method of determining the amplitude and/or phase of light using an optical detector on an application specific integrated circuit (ASIC), comprising:
driving a means for exciting a sample with an AC drive signal from a modulator;
exciting the sample with the means;
receiving with at least one photodiode light reflected by or emitted from or transmitted through the sample;
receiving at a lock in amplifier at least one diode signal from the at least one diode and a reference signal associated with the AC drive signal from the modulator; and
using lock-in detection to determine the phase and/or amplitude of the at least one diode signal from the at least one diode signal and the reference signal.

26. A method according to claim 25, wherein the step of driving comprises driving a light source with an AC drive signal from a light source modulator, and the step of exciting comprises illuminating the sample with the light source.

27. A method according to claim 25, wherein ASIC is housed in a product package having dimensions of about 2 mm×3 mm×1 mm.

28. A method according to claim 27, wherein the driving means for exciting the sample is located outside the product package and is driven by the ASIC.
